(19) United States
(12) Patent Application Publication
(10) Pub. No.: US 2020/0199685 A1
(43) Pub. Date: Jun. 25, 2020

(54) DETERMINATION OF A PHYSIOLOGICAL CONDITION WITH NUCLEIC ACID FRAGMENT ENDPOINTS

(71) Applicant: Guardant Health, Inc., Redwood City, CA (US)

(72) Inventors: Matthew William SNYDER, Seattle, WA (US); Jason Thaddeus DEAN, Seattle, WA (US)

(21) Appl. No.: 16/717,442

(22) Filed: Dec. 17, 2019

Related U.S. Application Data
(60) Provisional application No. 62/780,393, filed on Dec. 17, 2018.

(51) Int. Cl. C12Q 1/6886; G06F 17/18; G16B 50/30; G16B 50/10; G16B 45/00

(52) U.S. Cl. CPC C12Q 1/6886; G06F 17/18; G16B 50/30; G01N 2800/50; G16B 45/00; G01N 2800/52; G16B 50/10

(57) ABSTRACT

Methods for diagnosis of one or more physiological conditions using cfDNAs are disclosed. One embodiment of the invention is the computer implemented analysis of mapped circulating cell-free DNA fragment endpoint locations using a hidden Markov model to detect the presence of absence of cancer in a test subject. Another embodiment is a system for implementing the analysis of circulating cell-free DNA to detect the presence of absence of cancer using a hidden Markov model.

### CROSS-REFERENCE TO RELATED APPLICATION

This application claims the benefit of the priority date of U.S. Provisional Patent Application 62/780,393, filed Dec. 17, 2018, which is incorporated by reference herein in its entirety.

### FIELD

Provided are methods for diagnosis of physiological conditions, such as cancer, using cell-free DNA.

### BACKGROUND

Cell-free DNA (cfDNA) is present in the circulating plasma, urine, and other bodily fluids of humans. cfDNA contains both single- and double-stranded DNA fragments that are relatively short (overwhelmingly less than 200 base pairs) and are normally found at low concentrations in plasma (e.g. 1-100 ng/mL in plasma). In the circulating plasma of healthy individuals, cfDNA is believed to derive from apoptosis of blood cells, i.e. normal cells of the hematopoietic lineage. However, in specific situations, other tissues can contribute to cfDNA in plasma.

In recent years, efforts have been made to exploit cfDNA in conjunction with the emergence of new technologies related to cost-effective DNA sequencing in the development of diagnostics. In pregnant women, for example, a proportion of cfDNA in circulating plasma derives from fetal or placental cells. Screening for genetic abnormalities in the fetus, such as chromosomal trisomies, can be achieved by deep sequencing of the cfDNA of a pregnant woman, since the cfDNA of a pregnant woman is a mixture of cfDNA derived from the maternal and fetal genomes. One can expect to observe an excess of reads mapping to chromosome 21 if the fetus has trisomy 21. Non-invasive screening based on analysis of cfDNA is now routinely offered to pregnant women.

With respect to cancer diagnostics, a proportion of cfDNA in circulating plasma can come from a tumor, with the contribution from the tumor often increasing with cancer stage. Cancer is caused by abnormal cells exhibiting uncontrolled proliferation secondary to mutations in their genomes. The observation of cfDNA in circulating plasma has substantial promise to effectively serve as a diagnostic for cancer.

With respect to transplant rejection, after a transplant is performed, there is a risk of allograft rejection. Currently, the gold standard for assessing transplant rejection involves an invasive biopsy. A major challenge is determining whether and to what extent a rejection is occurring without an invasive biopsy. Recently, using cfDNA from the donor as a non-invasive marker for detecting allograft rejection has been explored.

There are several shared characteristics of current cfDNA diagnostic efforts. First, each relies on sequencing of cfDNA, generally from circulating plasma but potentially from other bodily fluids. Second, each relies on the fact that cfDNA comes from cell populations bearing genomes that differ very little from one another with respect to primary nucleotide sequence and/or copy number. Third, the basis for each is to detect or monitor genotypic differences between cell populations.

The reliance of cfDNA efforts in diagnostics on what are essentially genotypic differences is the basis of their success but also a major limitation. For example, since an overwhelming majority of cfDNA corresponds to regions of the human genome that are identical, the reliance on genotypic differences is uninformative when one is trying to discriminate between cell populations or between one group of subjects and another.

There is a need for a cfDNA test with greater discriminatory power.

### SUMMARY

The present application provides methods for identifying a physiological condition or diagnosing a disease, disorder, or condition in a subject by analysis of cfDNA fragments from a biological sample, specifically by applying a hidden Markov model to the frequency distribution of cfDNA fragment endpoint coordinates and assigning a diagnosis on the basis of the output from the model. In some embodiments, this disease is cancer. In some embodiments, the disease is lung adenocarcinoma, breast ductal carcinoma, or serous ovarian carcinoma.

A first aspect provides a method of identifying a physiological condition in a subject, the method comprising:
(a) providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
(b) providing at least one first training fragment endpoint map from at least one first reference sample from one or more subjects with at least one first physiological condition, the at least one first training fragment endpoint map comprising measured frequencies of the genomic locations of outer alignment coordinates, or a mathematical transformation thereof, within the reference genome for fragment endpoints from the at least one first reference sample;
(c) providing at least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample;
(d) training a hidden Markov model (HMM) with the at least one first training fragment endpoint map and the at least one second training fragment endpoint map;
(e) obtaining maximum likelihood estimates for hidden states at a plurality of genomic positions from the hidden Markov model for the sample;
(f) computing a summary statistic of the maximum likelihood estimates for the sample;
(g) comparing the summary statistic to a threshold value; and
(h) identifying the first physiological condition in the subject if the summary statistic exceeds the threshold value.

A second aspect of the invention provides a method of identifying or diagnosing a disease, disorder, or condition in a subject, the method comprising:
a. providing a testing fragment endpoint map from a sample from the subject, the testing fragment endpoint map comprising or consisting of measurements of the genomic locations of the outer alignment coordinates, or a mathematical transformation thereof, within a reference genome for at least some fragment endpoints;
b. providing at least one first training fragment endpoint map from at least one first reference sample from one or more subjects with a disease, disorder, or condition, the at least one first training fragment endpoint map comprising measured frequencies of the genomic locations of outer alignment coordinates, or
a mathematical transformation thereof, within the reference genome for fragment endpoints from the at least one first reference sample;
c. providing at least one second training fragment endpoint map from at least one second reference sample from subjects not having the disease, disorder, or condition, the at least one second training fragment endpoint map comprising measured frequencies of the genomic locations of outer alignment coordinates, or a mathematical transformation thereof, within the reference genome for fragment endpoints from the at least one second reference sample;
d. training a hidden Markov model with the at least one first training fragment endpoint map and the at least one second training fragment endpoint map;
e. obtaining maximum likelihood estimates for hidden states at a plurality of genomic positions from the hidden Markov model for the sample;
f. computing a summary statistic of the maximum likelihood estimates for the sample;
g. comparing the summary statistic to a threshold value; and
h. identifying or diagnosing the disease, disorder, or condition in the subject if the summary statistic exceeds the threshold value.

A third aspect of the invention provides a method of determining tissue(s) and/or cell type(s) giving rise to cfDNA in a subject, the method comprising:
(a) providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
(b) providing at least one first training fragment endpoint map for one or more subjects with at least one first physiological condition with tissue(s) and/or cell type(s) giving rise to fragment endpoints, the one first training fragment endpoint map comprising or consisting of measured frequencies of genomic locations of outer alignment coordinates, or a mathematical transformation thereof, of the fragment endpoints within the reference genome;
(c) providing at least one second training fragment endpoint map for one or more subjects with at least one second physiological condition with tissue(s) and/or cell type(s) giving rise to fragment endpoints, the at least one second training fragment endpoint map comprising or consisting of measured frequencies of genomic locations of outer alignment coordinates, or a mathematical transformation thereof, of fragment endpoints within a reference genome;
(d) training a hidden Markov model with the at least one first training fragment endpoint map and the at least one second training fragment endpoint map;
(e) obtaining maximum likelihood estimates for hidden states at a plurality of genomic positions from the hidden Markov model for the sample;
(f) computing a summary statistic of the maximum likelihood estimates for the sample;
(g) comparing the summary statistic to a threshold value; and
(h) determining tissue(s) and/or cell type(s) giving rise to fragment endpoints in the subject as being:

(i) from tissue(s) and/or cell type(s) associated with the at least one first physiological condition if the summary statistic exceeds a threshold value.

A fourth aspect provides a method of identifying at least one physiological condition in a subject, the method comprising:
(a) providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
(b) training a hidden Markov model with at least one first training endpoint map, the at least one first training endpoint map comprising or consisting of measured frequencies of the genomic locations of outer alignment coordinates, or a mathematical transformation thereof, within the reference genome for fragment endpoints from the at least one first reference sample and training the hidden Markov model with at least one second training endpoint map, the at least one second training endpoint map comprising or consisting of measured frequencies of the genomic locations of outer alignment coordinates, or a mathematical transformation thereof, within the reference genome for fragment endpoints from the at least one second reference sample;
(c) obtaining maximum likelihood estimates for hidden states at a plurality of genomic positions from the hidden Markov model for the sample;
(d) computing a summary statistic of the maximum likelihood estimates for the sample;
(e) comparing the summary statistic to a threshold value;
(f) identifying the physiological condition in the subject as the at least one physiological condition if the summary statistic exceeds a threshold value.

A fifth aspect provides a method of identifying at least one physiological condition in a subject, the method comprising:
(a) providing a fragment endpoint map from a sample from the subject, the fragment endpoint map comprising measurements of the genomic locations of the outer alignment coordinates, or a mathematical transformation, within a reference genome for at least some fragment endpoints;
(b) determining the physiological condition in the subject as the at least one first physiological condition in the subject if a summary statistic for the sample exceeds a threshold value, the summary statistic being computed from the maximum likelihood estimates for hidden states at a plurality of genomic positions from a hidden Markov model for the sample that has been trained with at least one first training fragment endpoint map and at least one second training fragment endpoint map, the at least one first and second training fragment endpoint maps comprising or consisting of measured frequencies of the genomic locations of outer alignment coordinates, or mathematical transformations thereof, within the reference genome for fragment endpoints from at least one first and at least one second reference sample, respectively.

A sixth aspect provides a method of recommending treatment for or providing treatment to a subject with a physiological condition in need thereof, the method comprising:
(a) providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
(b) providing at least one first training fragment endpoint map from at least one first reference sample from one or more subjects with at least one first physiological condition, the at least one first training fragment endpoint map comprising measured frequencies of the genomic locations of outer alignment coordinates, or a mathematical transformation thereof, within the reference genome for fragment endpoints from the at least one first reference sample;
(c) providing at least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample;
(d) training a hidden Markov model with the at least one first training fragment endpoint map and the at least one second training fragment endpoint map;
(e) obtaining maximum likelihood estimates for hidden states at a plurality of genomic positions from the hidden Markov model for the sample;
(f) computing a summary statistic of the maximum likelihood estimates for the sample;
(g) comparing the summary statistic to a threshold value;
(h) identifying the first physiological condition in the subject if the summary statistic exceeds the threshold value; and
(i) recommending treatment for or providing treatment to the subject for the first physiological condition.

A seventh aspect provides a method of training a hidden Markov model with at least one first training fragment endpoint map and at least one second training fragment endpoint map, the method comprising:
(a) providing at least one first training fragment endpoint map from at least one first reference sample from one or more subjects with at least one first physiological condition, the at least one first training fragment endpoint map comprising measured frequencies of the genomic locations of outer alignment coordinates, or a mathematical transformation thereof, within a reference genome for fragment endpoints from the at least one first reference sample;
(b) providing at the least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample; and
(c) training the hidden Markov model with the at least one first training fragment endpoint map and the at least one second training fragment endpoint map.

In some embodiments of any of the aspects provided herein, the fragment endpoints from the testing fragment endpoint map, the at least one first training endpoint map, and/or the at least one second training endpoint map comprise or consist of cfDNA fragment endpoints. In some embodiments, the second at least one physiological condition is a healthy human state. In some embodiments, the disease, disorder, or condition or at least one first physiological condition is cancer, normal pregnancy, a complication of pregnancy, myocardial infarction, inflammatory bowel disease, systemic autoimmune disease, localized autoimmune disease, allotransplantation with rejection, allotransplantation without rejection, stroke, and/or localized tissue damage. In some embodiments, the disease, disorder, or condition or at least one first physiological condition is cancer. In some embodiments, the disease, disorder, or condition or at least one physiological condition is lung adenocarcinoma, breast ductal carcinoma, or serous ovarian carcinoma. In some embodiments, the disease, disorder, or condition or at least one first physiological condition is colorectal cancer.

In some embodiments, the at least one first training fragment endpoint map and/or the at least one second training fragment endpoint map consist of positions or spacing of nucleosomes and/or chromatosomes, positions of transcription start sites and/or transcription end sites, positions of binding sites of at least one transcription factor, and/or positions of nuclease hypersensitive sites.

In some embodiments, the subject is human. In some embodiments, the subject is non-human. A human subject can be any gender, such as male or female. In some embodiments, the human can be an infant, child, teenager, adult, or elderly person. In some embodiments, the subject is a female subject who is pregnant, suspected of being pregnant, or planning to become pregnant.

In some embodiments, the subject is a mammal, a non-human mammal, a non-human primate, a primate, a domesticated animal (e.g., laboratory animals, household pets, or livestock), or a non-domesticated animals (e.g., wildlife). In some embodiments, the subject is a dog, cat, rodent, mouse, hamster, cow, bird, chicken, pig, horse, goat, sheep, rabbit, ape, monkey, or chimpanzee.

In some embodiments, the sample comprises or consists of whole blood, peripheral blood plasma, urine, or cerebral spinal fluid. In some embodiments, the sample comprises or consists of plasma samples.

In some embodiments, the at least one first training fragment endpoint map and/or the at least one second training fragment endpoint map comprises or consists of genomic positions or spacing of nucleosomes and/or chromatosomes, genomic positions of transcription start sites and/or transcription end sites, genomic positions of binding sites of at least one transcription factor, and/or genomic positions of nuclease hypersensitive sites. In some embodiments, the subject is human.

In some embodiments, the disease, disorder, or condition, at least one first physiological condition, and/or at least one second physiological condition is healthy. In some embodiments, the disease, disorder, or condition, at least first physiological condition, and/or at least one second physiological condition is selected from the group consisting of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage.

In some embodiments, the disease, disorder, or condition, at least one first physiological condition, and/or at least one second physiological condition is cancer. In some embodiments, the disease, disorder, or condition, at least one first physiological condition, and/or at least one second physiological condition is lung adenocarcinoma, breast ductal carcinoma, or serous ovarian carcinoma. In some embodiments, the disease, disorder, or condition, at least one first physiological condition, and/or at least one second physiological condition is colorectal cancer. In some embodiments, the disease, disorder, or condition, at least one first physiological condition, and/or at least one second physiological condition is selected from the group consisting of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage.

In some embodiments, at least some of the cfDNA fragments are subjected to a size selection to retain only cfDNA fragments having a length between an upper bound and a lower bound. In some embodiments, the upper bound is about 200, about 190, about 180, about 170, about 160, about 150, about 140, about 130, about 120, about 110, about 100, about 90, about 80, about 70, about 60, or about 50 base pairs and the lower bound is about 20, about 25, about 30, about 35, about 36, about 40, about 45, about 50, about 60, about 70, about 80, about 90, about 100, about 110, or about 120 base pairs.

In some embodiments, a subset of isolated cfDNA fragments from the subject is targeted for sequencing on the basis of genomic locations and/or annotations. In some embodiments, the subset is targeted to transcription start sites (TSSs).

In some embodiments, the method further comprises generating a report listing a plurality of probability scores calculated for the biological sample from the subject using either or both of the at least one first training sample and/or the at least one second training sample. In some embodiments, the method any of the above claims further comprises recommending treatment for the identified disease or condition in the subject. In some embodiments, the method further comprises treating the identified condition in the subject.

In other aspects, the present disclosure provides a system, comprising a controller comprising, or capable of accessing, computer readable media comprising non-transitory computer-executable instructions which, when executed by at least one electronic processor perform at least: generating at least one first training fragment endpoint map from at least one first reference sample from one or more subjects with at least one first physiological condition, the at least one first training fragment endpoint map comprising measured frequencies of genomic locations of outer alignment coordinates, or a mathematical transformation thereof, within a reference genome for fragment endpoints from the at least one first reference sample; generating at least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample; and training a hidden Markov model with the at least one first training fragment endpoint map and the at least one second training fragment endpoint map.

In still other aspects, the present disclosure provides a computer readable media comprising non-transitory computer-executable instructions which, when executed by at least one electronic processor perform at least: generating at least one first training fragment endpoint map from at least one first reference sample from one or more subjects with at least one first physiological condition, the at least one first training fragment endpoint map comprising measured frequencies of genomic locations of outer alignment coordinates, or a mathematical transformation thereof, within a reference genome for fragment endpoints from the at least one first reference sample; generating at least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample; and training a hidden Markov model with the at least one first training fragment endpoint map and the at least one second training fragment endpoint map.

In some embodiments of the systems and computer readable media disclosed herein, the instructions further perform at least: generating a testing fragment endpoint map from a test sample from a test subject, the testing fragment endpoint map comprising measurements of the genomic locations of the outer alignment coordinates, or a mathematical transformation thereof, within the reference genome for at least some fragment endpoints. In certain embodiments of the systems and computer readable media disclosed herein, the instructions further perform at least: obtaining maximum likelihood estimates for hidden states at a plurality of genomic positions from the hidden Markov model for the test sample. In some embodiments of the systems and computer readable media disclosed herein, the instructions further perform at least: computing at least one summary statistic of the maximum likelihood estimates for the test sample. In certain embodiments of the systems and computer readable media disclosed herein, the instructions further perform at least: comparing the summary statistic to a threshold value. In some embodiments of the systems and computer readable media disclosed herein, the instructions further perform at least: identifying the at least one first physiological condition in the test subject if the summary statistic exceeds the threshold value. In certain embodiments of the systems and computer readable media disclosed herein, the instructions further perform at least: recommending treatment for the test subject for the first physiological condition.

### BRIEF DESCRIPTION OF THE FIGURES

FIG. 1 is a schematic diagram of an exemplary system suitable for use with certain aspects disclosed herein.

FIG. 2 depicts the first two principal components of the matrix resulting from vectors of hidden Markov model output for each sample with each column representing results for a single sample and rows representing the mean of the results for each region. The x-axis shows principal component 1 and the y-axis shows principal component 2.

FIG. 3 depicts scores resulting from an estimate for blinded samples used to generate a prediction. Each column in the x-axis shows a sample and the y-axis shows the estimated probability of cancer from a trained support vector machine.

FIG. 4 depicts the first two principal components for vectors of hidden Markov model output combined into a matrix. The x-axis shows principal component 1 and the y-axis shows principal component 2.

FIG. 5 depicts the first two principal components for vectors of hidden Markov model output combined into a matrix. The x-axis shows principal component 1 and the y-axis shows principal component 2.

FIG. 6 depicts LD1 scores for solid tumor types, stratified by stage. The x-axis shows stage and the y-axis shows LD1 score.

FIG. 7 depicts LD1 scores for healthy controls. The x-axis shows a healthy stage and the y-axis shows LD1 score.

FIG. 8 depicts the receiver operating characteristic curve for each tumor type, determined by sliding classification decision boundary stepwise from the minimum to the maximum LD1 score and calculating the sensitivity and specificity of each resulting hypothetical classifier. The x-axis shows 1-specificity and the y-axis shows sensitivity.

### DESCRIPTION OF EXEMPLARY EMBODIMENTS

The present application provides methods for identifying a physiological condition or diagnosing a disease, disorder, or condition in a subject by analysis of cfDNA fragments from a biological sample, specifically by applying a hidden Markov model to the frequency distribution of cfDNA fragment endpoint coordinates and assigning a diagnosis on the basis of the output from the model. In some embodiments, this disease is cancer. In some embodiments, the disease is lung adenocarcinoma, breast ductal carcinoma, or serous ovarian carcinoma. In some embodiments, the disease is colorectal cancer.

### I. Definitions

As herein, the term “about” when referring to a number or a numerical range means that the number or numerical range referred to is an approximation within experimental variability (or within statistical experimental error), and the number or numerical range may vary from, for example, from 1% to 15% of the stated number or numerical range.

As used herein, “allotransplantation” refers to the transplantation of cells, tissues, or organs to a recipient from a genetically non-identical donor of the same species. The transplant is called an allograft, allogeneic transplant, or homograft. Most human tissue and organ transplants are allografts.

As used herein, “annotations,” “DNA annotations,” “genome annotation,” or “genomic annotations” refer to the locations of genes, coding regions, and functional areas and the determination of what those genes, coding regions, and functional areas do.

As used herein, “autoimmune disease” refers to a condition resulting from an abnormal immune response to a normal body part.

As used herein, “burden” refers to a load or weight with respect to a particular disease or physiological condition. In particular, a burden is normally used to indicate an increased load or weight of a disease or physiological condition.

As used herein, “cancer” refers to disease caused by an uncontrolled division of abnormal cells in a part of the body.

As used herein, “cell-free DNA” or “cfDNA” refers to DNA fragments present in the blood plasma.

As used herein, “fragment endpoints” or “endpoints” shall refer to the termini of cfDNA.

As used herein, “fragment endpoint map” and “fragment endpoint profile” shall mean the same thing.

As used herein, “genome” or “genomic” refers to the complete set of genes or genetic material present in a cell or organism.

As used herein, “healthy” refers to a subject, such as a human, that does not have a disease, disorder, or condition. A healthy subject shall be one that does not have a considered or specified disease, disorder, or condition and the term healthy, as used herein, shall be used with respect to the considered or specified disease, disorder, or condition as a subject that does not have the considered or specified disease, disorder, or condition, despite having another or some other disease, disorder, or condition that does not relate to the considered or specified disease, disorder, or condition.

As used herein, “hidden Markov model” or “HMM” refers a statistical Markov model in which the system being modeled is assumed to be a Markov process with unobserved (i.e. hidden) states. A hidden Markov model can be represented as the simplest dynamic Bayesian network (See, Baum, L. E.; Petrie, T. (1966). Statistical Inference for Probabalistic Functions of Finite State Markov Chains. The Annals of Mathematical Statistics. 37 (6): 1554-1563, 28 Nov. 2011, which is incorporated by reference herein in its entirety, including any drawings).

As used herein, “inflammatory bowel disease” refers to group of chronic intestinal diseases characterized by inflammation of the bowel in the large or small intestine. The most common types of inflammatory bowel disease are ulcerative colitis and Crohn's disease.

As used herein, “mathematical transformation” refers to a function, ƒ that maps a set X to itself such as ƒ:X→X. A transformation may simply be any function, regardless of domain and codomain. Examples include linear transformations and affine transformations, rotations, reflections, and translations. Examples of transformations include, without limitation, a Fourier transformation, a fast Fourier transformation, and/or a window protection score.

As used herein, “myocardial infarction” refers to the irreversible death or necrosis of heart muscle secondary to prolonged lack of oxygen supply.

As used herein, “next generation sequencing” refers to any high-throughput sequencing approach including, but not limited to, one or more of the following: massively-parallel signature sequencing, pyrosequencing (e.g., using a Roche 454 sequencing device), Illumina sequencing, sequencing by synthesis, ion torrent sequencing, sequencing by ligation (“SOLiD”), single molecule real-time (“SMRT”) sequencing, colony sequencing, DNA nanoball sequencing, heliscope single molecule sequencing, and nanopore sequencing.

As used herein, “peripheral blood” refers to the flowing, circulating blood of the body. It is normally composed of erythrocytes, leukocytes, and thrombocytes. These blood cells are suspended in blood plasma, through which the blood cells are circulated through the body. Peripheral blood is different from the blood whose circulation is enclosed within the liver, spleen, bone marrow, and the lymphatic system. These areas contain their own specialized blood.

As used herein, “peripheral blood plasma” refers to the plasma found in peripheral blood.

As used herein, “plasma” or “blood plasma” refers to the liquid component of blood that normally holds the blood cells in whole blood in suspension. Holding blood cells in whole blood makes plasma the extracellular matrix of blood cells.

As used herein, “stroke” refers to the sudden death of brain cells due to lack of oxygen caused by blockage of blood flow or rupture of an artery to the brain.

As used herein, “threshold value” refers to a summary statistic value chosen such that a certain percentage of values determined for the at least one first training fragment endpoint map are above the threshold value and/or a certain percentage of values determined for the at least one second training fragment endpoint map are below the threshold value. For example, a threshold value may be chosen such that at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 97%, at least about 98%, or at least about 99% of values determined for the at least one first training fragment endpoint map are above the threshold value and/or at least about 60%, at least about 62%, at least about 64%, at least about 66%, at least about 68%, at least about 70%, at least about 72%, at least about 74%, at least about 76%, at least about 78%, at least about 80%, about 82%, at least about 84%, at least about 86%, at least about 88%, at least about 90%, at least about 92%, at least about 94%, at least about 96%, at least about 97%, at least about 98%, or at least about 99% of values determined for the at least one second training fragment endpoint map are below the threshold value. A threshold value may be determined according to one skilled in the art or as set forth in the examples.

As used herein, “whole blood” refers to blood drawn directly from the body from which no components, such as plasma or platelets, have been removed.

As used herein, “windowed protection score,” “window protection score,” or “WPS” refers to the number gained by subtracting the number of fragment endpoints within a 120 bp window from the number of fragments completely spanning the window (See, for example, W02016015058A2, which is incorporated in its entirety herein, including any drawings).

### II. Subjects

A subject may be any subject known to one skilled in the art. In some embodiments, the subject is human. In some embodiments, the subject is non-human. A human subject can be any gender, such as male or female. In some embodiments, the human can be an infant, child, teenager, adult, or elderly person. In some embodiments, the subject is a female subject who is pregnant, suspected of being pregnant, or planning to become pregnant.

In some embodiments, the subject is a mammal, a non-human mammal, a non-human primate, a primate, a domesticated animal (e.g., laboratory animals, household pets, or livestock), or a non-domesticated animals (e.g., wildlife). In some embodiments, the subject is a dog, cat, rodent, mouse, hamster, cow, bird, chicken, pig, horse, goat, sheep, rabbit, ape, monkey, or chimpanzee.

### III. Biological Samples

Biological samples can be any type known to one skilled in the art and may be obtained from any subject. In some embodiments, the biological sample is from a human subject. In some embodiments, the biological sample is from a non-human subject. In some embodiments, a biological sample is isolated from one or more subjects having one or more physiological conditions. In some embodiments, the one or more physiological conditions are one or more healthy human states and/or human disease states.

In some embodiments, biological samples comprise or consist of unprocessed samples (e.g., whole blood, tissue, or cells) or processed samples (e.g., serum or plasma). In some embodiments, biological samples are enriched for a certain type of nucleic acid. In some embodiments, biological samples are processed to isolate nucleic acids from other components within the biological sample.

In some embodiments, biological samples comprise cells, tissue, a bodily fluid, or a combination thereof. In some embodiments, biological samples comprise or consist of whole blood, peripheral blood plasma, urine, or cerebral spinal fluid. In some embodiments, biological samples comprise or consist of a blood components, plasma, serum, synovial fluid, bronchial-alveolar lavage, saliva, lymph, spinal fluid, nasal swab, respiratory secretions, stool, peptic fluids, vaginal fluid, semen, and/or menses.

In some embodiments, biological samples comprise or consist of fresh samples. In some embodiments, biological samples comprise or consist of frozen samples. In some embodiments, biological samples comprise fixed samples, e.g., samples fixed with a chemical fixative such as formalin-fixed paraffin-embedded tissue.

Biological samples may also be obtained at any point during medical care. In some embodiments, biological samples are obtained prior to treatment, during the treatment process, after diagnosis, or any other point. Biological samples may be obtained at specific intervals, such as daily, weekly, or monthly, or during a routine medical examination.

### IV. Isolating cfDNA

Isolation of cfDNA can proceed according to any method known to those of skill in the art. For example, the QIAGEN QlAamp Circulating Nucleic Acid kit is commonly used to isolate cfDNA from plasma or urine based on binding of cfDNA to a silica column. Isolation may also include phenol-chloroform extraction followed by isopropanol or ethanol precipitation.

In some embodiments, isolating cfDNA is done in such a manner as to maximize the recovery of short fragments (<100 base pairs), as the composition of short fragments differs more strongly between healthy and disease states than the composition of longer fragments does between healthy and disease samples. In some embodiments, any of the cfDNA fragments are subjected to a size selection to retain only cfDNA fragments having a length between an upper bound and a lower bound. In some embodiments, the upper bound is about 200, about 190, about 180, about 170, about 160, about 150, about 140, about 130, about 120, about 110, about 100, about 90, about 80, about 70, about 60, or about 50 base pairs and the lower bound is about 20, about 25, about 30, about 35, about 36, about 40, about 45, about 50, about 60, about 70, about 80, about 90, about 100, about 110, or about 120 base pairs. In some embodiments, the lower bound is 36 and the upper bound is 100.

### V. Constructing a Sequencing Library

After isolating cfDNA from a biological sample, isolated cfDNA comprising a plurality of cfDNA fragments can be subjected to one or more enzymatic steps to create a sequencing library. Enzymatic steps can proceed according to techniques known to those of skill in the art. Enzymatic steps may include 5′ phosphorylation, end repair with a polymerase, A-tailing with a polymerase, ligation of one or more sequencing adapters with a ligase, and linear or exponential amplification with a polymerase.

Preparation of sequencing libraries may be performed to maximize the conversion of short fragments (<100 base pairs). In some embodiments, a physical size-selection step is employed to select for short cfDNA fragments. In some embodiments, an enrichment step is employed, wherein the enrichment step comprises enriching cfDNA that are targeted to a genomic location. An enrichment step may be employed by itself or in conjunction with a physical size-selection step. A physical size selection step could comprise or consist of gel electrophoresis and/or capillary electrophoresis. In some embodiments, constructing a sequencing library should preserve the original termini of cfDNA fragments.

Some embodiments comprise attaching adapters to the plurality of cfDNA fragments to aid in purification, detection, amplification, or a combination thereof. In some embodiments, the adapters are sequencing adapters. In some embodiments, at least some of the plurality of cfDNA fragments are attached to the same adapter. In some embodiments, different adaptors are attached at both ends of the plurality of cfDNA fragments. In some embodiments, at least some of the plurality of cfDNA fragments may be attached to one or more adapters on one end. Adapters may be attached to cfDNAs by primer extension, reverse transcription, or hybridization.

In some embodiments, an adapter is attached to a plurality of cfDNA fragments by ligation. In some embodiments, an adapter is attached to a plurality of cfDNA fragments by a ligase. In some embodiments, an adapter is attached to a plurality of cfDNA fragments by sticky-end ligation or blunt-end ligation. An adapter may be attached to the 3′ end, the 5′ end, or both ends of the plurality of cfDNA fragments.

In some embodiments, enzymatic end-repair processes are used for adapter ligation. The end repair reaction may be performed by using one or more end repair enzymes (e.g., a polymerase and an exonuclease).

In some embodiments, the ends of the plurality of cfDNA fragments can be polished by treatment with a polymerase. Polishing can involve removal of 3′ overhangs, fill-in of 5′ overhangs, or a combination thereof. For example, a polymerase may fill in the missing bases for a DNA strand from 5′ to 3′ direction. The polymerase can be a proofreading polymerase (e.g., comprising 3′ to 5′ exonuclease activity). The proofreading polymerase can be, e.g., a T4 DNA polymerase, Pol 1 Klenow fragment, or Pfu polymerase. Polishing can comprise removal of damaged nucleotides using any means known in the art. In some embodiments, the ends of the plurality of cfDNA fragments are polished by treatment with an exonuclease to remove the 3′ overhangs.

### VI. Sequencing of Fragment Endpoints

In some embodiments, sequencing fragment endpoints of the plurality of cfDNA fragments comprises or consists of sequencing the plurality of cfDNA fragments. In some embodiments, sequencing fragment endpoints of the plurality of cfDNA fragments comprises or consists of sequencing an entire cfDNA fragment(s) of the plurality of cfDNA fragments. In some embodiments, sequencing fragment endpoints of the plurality of cfDNA fragments comprises or consists of sequencing only the fragment endpoints of the plurality of cfDNA fragments.

Following the preparation of a sequencing library, at least the fragment endpoints of the plurality of cfDNA fragments are sequenced. Any method known to one skilled in the art may be used to generate a dataset consisting of at least one “read” (the ordered list of nucleotides comprising each sequenced molecule). In some embodiments, sequencing fragment endpoints comprises or consists of next generation sequencing assay.

In some embodiments, sequencing comprises or consists of classic Sanger sequencing methods that are well known in the art. In some embodiments, sequencing comprises or consists of sequencing on an Illumina Novaseq instrument with an S4 flow cell. In some embodiments, sequencing comprises or consists of sequencing on Illumina's Genome Analyzer IIX, MiSeq personal sequencer, NextSeq series, or HiSeq systems, such as those using HiSeq 4000, HiSeq 3000, HiSeq 2500, HiSeq 1500, HiSeq 2000, or HiSeq 1000. In some embodiments, sequencing comprises or consists of using technology available by 454 Lifesciences, Inc. to sequence fragment endpoints. In some embodiments, sequencing comprises or consists of ion semiconductor sequencing (e.g., using technology from Life Technologies (Ion Torrent)).

In some embodiments, sequencing comprises or consists of nanopore sequencing (See e.g., Soni GV and Meller A. (2007) Clin Chem 53: 1996-2001, which is incorporated by reference in its entirety, including any drawings). In some embodiments, nanopore sequencing comprises or consists of using technology from Oxford Nanopore Technologies; e.g., a GridION system. In some embodiments, nanopore sequencing comprises or consists of strand sequencing in which intact DNA polymers can be passed through a protein nanopore with sequencing in real time as the DNA translocates the pore.

In some embodiments, nanopore sequencing comprises or consists of exonuclease sequencing in which individual nucleotides can be cleaved from a DNA strand by a processive exonuclease and the nucleotides can be passed through a protein nanopore. In some embodiments, nanopore sequencing comprises or consists of nanopore sequencing technology from GENIA. In some embodiments, nanopore sequencing comprises or consists of technology from NABsys. In some embodiments, nanopore sequencing comprises or consists of technology from IBM/Roche.

In some embodiments, sequencing comprises or consists of sequencing by ligation approach. One example is the next generation sequencing method of SOLiD sequencing. SOLiD may generate hundreds of millions to billions of small sequence reads at one time.

### VII. Determining a Genomic Location of Fragment Endpoints

For each dataset (i.e., for each sequenced library of a plurality of fragment endpoints), the two genomic endpoints of each sequenced fragment endpoints are identified with computer software. After sequencing of cfDNA fragments and fragment endpoints and appropriate quality control, a genomic location for the fragment endpoints within a reference genome is determined. The process of determining genomic locations, or mapping, identifies the genomic origin of each fragment based on a sequence comparison, determining, for example, that a given fragment of cfDNA was originally part of a specific region of chromosome 12. Determining a genomic location of fragment endpoints can be done with any human reference genome, such as, for example, Genbank hg19 or Genbank hg38, using bwa software (See, http://bio-bwa.sourceforge.net/. which is incorporated by reference herein; See, WO 2016/015058, which is incorporated by reference herein in its entirety, including any drawings).

The procedure is performed for each library derived from each biological sample to produce one dataset per library. The procedure of mapping provides two fragment endpoints for each cfDNA fragment. The fragment endpoints are given numerical values (“coordinates”), representing the specific offset, relative to one end of a chromosome, of the fragment endpoint's location within the reference genome.

Fragment endpoints are the genomic coordinates, within a reference genome, of the two ends of each sequenced fragment. In some embodiments, fragment endpoints are determined by the process of mapping a fragment to a reference genome by means of a computer program, and obtaining the genomic coordinates of the two ends of the fragment by extracting the least and greatest numerical coordinates in the reference genome corresponding to the determined origin of the fragment. In some embodiments, fragment endpoints are determined by aligning or mapping the one or more reads from a fragment against a reference genome by means of a computer program, and obtaining the left-most and right-most (or least and greatest) outer alignment coordinates in the reference genome for the one or more reads corresponding to the fragment.

In some embodiments, fragment endpoints are further oriented in two dimensions, such that for every fragment endpoint, a given fragment endpoint's coordinate is either greater than or less than its partner's coordinate. In other words, each fragment endpoint is the left-most or right-most fragment endpoint coordinate of the pair in two-dimensional space. In some embodiments, a plurality of the fragment endpoints are classified based on the strand, for example Watson or Crick, from which their associated, sequenced cfDNA fragment was derived.

In the case of paired-end sequencing, the genomic coordinates of both fragment endpoints are inferred from mapping or alignment of the reads to the reference genome and are extracted by means of a computer program. In the case of single-end sequencing in which the entire fragment is sequenced (i.e., where the read length is equal to or greater than the length of the original fragment), the genomic coordinates of both fragment endpoints are inferred from mapping or alignment to the reference genome and are extracted by means of a computer program. In the case of single-end sequencing in which the entire fragment is not sequenced (i.e., where the read length is shorter than the original fragment), the genomic coordinate of only one endpoint is inferred from alignment to the reference genome and is extracted by means of a computer program.

In some embodiments, the genomic location of the first fragment endpoints and the second reference fragment endpoints may be determined with an available database. In some embodiments, the available database comprises or consists of a public database.

The method according to the invention may be shortened when using an available database. When using an available database, some embodiments comprise a method for detecting and/or diagnosing a disease or physiological condition in a subject in need thereof, comprising:
a. determining genomic locations of first fragment endpoints within a reference genome using available database fragment endpoints, the first fragment endpoints corresponding to at least one first physiological condition;
b. determining at least one first training fragment endpoint map for the first fragment endpoints;
c. determining genomic locations of second fragment endpoints within a reference genome using available database fragment endpoints, the second fragment endpoints corresponding to at least one second physiological condition;
d. determining at least one second training fragment endpoint map for the second fragment endpoints;
e. isolating cfDNA from a biological sample from the subject, the cfDNA comprising a sample plurality of cfDNA fragments;
f. constructing a sample sequencing library from the sample plurality of cfDNA fragments;
g. sequencing sample fragment endpoints of the sample plurality of cfDNA fragments;
h. determining genomic locations of the sample fragment endpoints within the reference genome for at least some of the sample plurality of cfDNA fragments as a function of the sequences;
i. training a hidden Markov model with the at least one first training fragment endpoint map and the at least one second training fragment endpoint map;
j. obtaining maximum likelihood estimates for hidden states at a plurality of genomic positions from the hidden Markov model for the sample;
k. computing a summary statistic of the maximum likelihood estimates for the sample;
l. comparing the summary statistic to a threshold value; and
m. identifying the physiological condition as the at least one first physiological condition in the subject if the summary statistic exceeds the threshold value.

### VIII. Constructing Testing and Training Datasets

The fragment endpoints are tallied at each of one or more specified coordinates in the reference genome to create one or more vectors of endpoint counts, where each item in each vector records the number of endpoints observed at a given genomic coordinate. In some embodiments, one vector is produced for each of a list of specified genomic regions, where each region can be of arbitrary size. In other embodiments, one vector is produced for each chromosome or chromosome arm in the reference genome. In other embodiments, one vector is produced for the entire reference genome.

The set of genomic coordinates represented in the one or more vectors produced for each training cfDNA sequencing dataset is either a superset of, or an identical set to, the set of genomic coordinates represented in the one or more vectors produced for the test cfDNA sequencing dataset.

Vectors are determined with the number of fragment endpoints observed at each genomic location. Some embodiments comprise a set of two or more vectors, each having a single entry for a single coordinate under consideration. In some embodiments, for example, the physiological conditions comprise a healthy human state. In some embodiments, the physiological conditions comprise a human disease state.

Within each vector, integer counts at each coordinate are converted to relative frequencies by dividing each integer count value by the sum of all integer count values in a vector. For example, if the sum of all integer counts in a vector is 1000, and the first three coordinates in the vector have integer counts of 1, 4, and 0, the resulting relative frequencies will be 1/1000, 4/1000, and 0/1000, respectively. The process is repeated for each vector representing each physiological condition. The resulting relative frequency values for the given set of coordinates and for a physiological condition comprise a vector for the physiological condition.

In some embodiments, the set of two or more vectors are visualized. In some embodiments, the set of two of more vectors are visualised as a two-dimensional histogram or scatterplot.

In some embodiments, vectors are normalized to correct for differences in sequencing depth or coverage, fragment length distribution, local GC content, and chromosome number between the at least one first physiological condition, the at least one second physiological condition, and the subject. Normalization can be performed using standard techniques known to those skilled in the art.

In some embodiments, one or more of the produced vectors may be subjected to one or more steps to produce a modified vector. In some embodiments, the vector may be normalized or downsampled by means of a computer program, such that the vector sum is a specified constant C. If C is 1, the vector represents a frequency vector, such that the value at each position in the vector represents the frequency, relative to all genomic coordinates represented in the vector, at which endpoints are observed at said position. In another example, the vector may be smoothed or de-noised, for example with a Gaussian kernel, by means of a computer program. In some embodiments, values of 0, representing coordinates at which no fragment endpoints were observed, are changed to a small number in order to enable downstream calculations that would otherwise be undefined, owing to potential division by zero or other considerations.

Construction of training datasets are very related to the construction of the testing datasets. Separately for each group of individuals sharing a common diagnosis, the set of vectors is combined across the one or more members of the group to create a training dataset for a given diagnosis. The method of combining vectors may be, in some embodiments, the calculation of the mean value at each vector position. In other embodiments, the median value at each vector position is calculated. In other embodiments, the sum of the vectors is calculated.

Training samples can be treated as both training and test samples, the training samples being treated as training samples initially and test samples subsequently. For example, a model may be trained with two sets of training samples and then each of the training samples can be run through the model to calculate the summary statistic from the output. Using training samples as both training samples and test samples may be used to assist in the determination of threshold values.

Alternatively, another set of samples with known labels, such as those not used for training, may be used to assist in the determination of the threshold value for a first round of testing. For example, one could use some proportion of training samples, such as half, for training a hidden Markov model and use the rest of the proportion for a first round of testing with the trained model.

Some embodiments provide for a method of training a hidden Markov model with at least one first training fragment endpoint map and at least one second training fragment endpoint map, the method comprising:
(a) providing the at least one first training fragment endpoint map from at least one first reference sample from one or more subjects with at least one first physiological condition, the at least one first training fragment endpoint map comprising measured frequencies of the genomic locations of outer alignment coordinates, or a mathematical transformation thereof, within a reference genome for fragment endpoints from the at least one first reference sample;
(b) providing at the least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample; and
(c) training the hidden Markov model with the at least one first training fragment endpoint map and the at least one second training fragment endpoint map.

### IX. Selecting Fragment Endpoints and Genomic Annotations

In some embodiments, sequenced reads are subjected to one or more filtering steps prior to the determination of endpoint coordinates. For example, reads may be discarded if the mapping quality of the reads is below a threshold value. An example threshold value for a mapping quality filter is 60.

In some embodiments, reads may be retained or discarded on the basis of the inferred length of the associated cfDNA fragment. For example, reads may be retained when corresponding to fragments having an inferred length above a specified threshold value, below a specified threshold value, or both; and may be preferentially discarded when not meeting the specified criteria. As an example, those fragments with lengths greater than or equal to 120 base-pairs (bp) are retained and those with lengths below 120 bp are discarded. In another example, those fragments having lengths between 36 and 100 bp (inclusive) are retained, and those fragments shorter than 36 bp or longer than 100 bp are discarded. These filtering steps are performed by means of one or more computer programs.

In some embodiments, the method further comprises filtering isolated cfDNA to retain cfDNA having a length between an upper bound and a lower bound. In some embodiments, the upper bound is about 200, about 190, about 180, about 170, about 160, about 150, about 140, about 130, about 120, about 110, about 100, about 90, about 80, about 70, about 60, or about 50 base pairs and the lower bound is about 20, about 25, about 30, about 35, about 36, about 40, about 45, about 50, about 60, about 70, about 80, about 90, about 100, about 110, or about 120 base pairs. In some embodiments, only fragments falling within a specified length range, such as 36-100 base pairs, are retained. In some embodiments, filtering comprises gel electrophoresis and/or capillary electrophoresis.

In some embodiments, a subset of isolated cfDNA is targeted to a genomic location. In some embodiments, a subset of isolated cfDNA fragments from the subject is targeted for sequencing on the basis of genomic locations and/or annotations. In some embodiments, the subset is targeted to transcription start sites (TSSs).

In some embodiments, the genomic location comprises one or more genomic annotations. In some embodiments, the one or more genomic annotations comprises DNA-binding or DNA-contacting proteins.

Genomic annotations enrich genomic locations by providing functional information related to location in the genome. Once a genome is sequenced it can be annotated to make sense of it. For DNA annotation, a previously unknown sequence of genetic material is enriched with information relating genomic position to intron-exon boundaries, regulatory sequences, repeats, gene names, and protein products. The National Center for Biomedical Ontology (www.bioontology.org) develops tools for annotation of database records based on the textual descriptions of those records.

In some embodiments, the one or more genomic annotations comprises or consists of transcription start sites. A transcription start site is the location where transcription starts at the 5′-end of a gene sequence. As the starting place for transcription, proteins involved in transcription may be expected to affect and influence fragment endpoints, especially between one physiological condition and another.

In some embodiments, the one or more genomic annotations comprises or consists of nucleosomes. Nucleosomes are known to be positioned in relation to landmarks of gene regulation, for example transcriptional start sites and exon-intron boundaries.

### X. Physiological States and Conditions

In some embodiments, cfDNA is isolated for the disease, disorder, or condition, at least one first physiological condition and/or at least one second physiological condition. The disease, disorder, or condition, at least one first physiological condition, and/or at least one second physiological condition comprise one or more healthy states or one or more disease states. In some embodiments, the one or more disease states comprise or consist of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage.

In some embodiments, the at least one first physiological condition and/or at least one second physiological condition comprises or consists of cancer. In some embodiments, cancer comprises or consists of acute lymphoblastic leukemia; acute myeloid leukemia; acute myeloid leukemia; adrenocortical carcinoma; AIDS-Related cancers; anal cancer; astrocytomas; central nervous system cancers; basal cell carcinoma; bile duct cancer; bladder cancer; bone cancers; brain stem glioma; brain tumors; craniopharyngioma; ependymoblastoma; medulloblastoma; medulloepithelioma; pineal parenchymal tumors; neuroectodermal tumors; breast cancer; bronchial tumors; Burkett's lymphoma; gastrointestinal cancers; cervical cancers; chronic lymphocytic leukemia; chronic myelogenous leukemia; chronic myeloproliferative disorders; colon cancer; colorectal cancer; cutaneous T-Cell lymphomas; endometrial cancers; esophageal cancers; Ewing cancers; extracranial germ cell tumors; eye cancers; retinoblastoma; gallbladder cancers; gastric cancers; gastrointestinal stromal tumor (GIST); ovarian cancers; hairy cell leukemia; head and neck cancer; heart cancer, hepatocellular cancers; Hodgkin's lymphoma; Kaposi's sarcoma; kidney cancers; lip and oral cavity cancers; liver cancers; lung cancers; non-small cell lung cancer; lymphoma; Waldenstrom macroglobulinemia; melanomas; mesothelioma; metastatic squamous neck cancers; mouth cancers; nasopharyngeal cancers; neuroblastoma; ovarian cancers; pancreatic cancer; penile cancers; pituitary tumors; rectal cancers; salivary gland cancers; squamous cell carcinomas; stomach cancers; throat cancers; thyroid cancers; and vaginal cancers. In some embodiments, cancer consists of lung adenocarcinoma, breast ductal carcinoma, or serous ovarian carcinoma. In some embodiments, cancer consists of colorectal cancer.

In some embodiments, at least one first physiological condition consists of a cancer at a first clinical stage (e.g., stage I) and the at least one second physiological condition consists of a cancer at a second clinical stage (e.g., stage IV). In some embodiments, the first clinical stage consists of a cancer at stage 0, stage I, stage II, stage III, or stage IV. In some embodiments, the second clinical stage consists of a cancer at stage 0, stage I, stage II, stage III, or stage IV.

In some embodiments, the disease, disorder, or condition, at least one first physiological condition, and/or at least one second physiological condition comprises or consists of normal pregnancy or complications of pregnancy. In some embodiments, the disease, disorder, or condition, at least one first physiological condition, and/or at least one second physiological condition comprises or consists of myocardial infarction or inflammatory bowel disease. In some embodiments, the disease, disorder, or condition, at least one first physiological condition, and/or at least one second physiological condition comprises or consists of allotransplantation with rejection and/or allotransplantation without rejection.

### XI. Obtaining Maximum Likelihood Estimates for Hidden States at a Plurality of Genomic Positions from the Hidden Markov Model

Some embodiments comprise or consist of obtaining maximum likelihood estimates for hidden states at a plurality of genomic positions from the hidden Markov model. A hidden Markov model is used as a generative model that emits endpoint counts at one or more coordinates, conditional on model parameters. A hidden Markov model is a statistical Markov model in which a system being modeled is assumed to be a Markov process with unobserved (i.e. hidden) states (See, Baum, L. E.; Petrie, T. (1966). Statistical Inference for Probabalistic Functions of Finite State Markov Chains. The Annals of Mathematical Statistics. 37 (6): 1554-1563, 28 Nov. 2011, which is incorporated by reference herein in its entirety, including any drawings). The hidden Markov model can be represented as the simplest dynamic Bayesian network. In simpler Markov models (like a Markov chain), the state is directly visible to the observer, and therefore the state transition probabilities are the only parameters, while in the hidden Markov model, the state is not directly visible, but the output (in the form of data or “token” in the following), dependent on the state, is visible. Each state has a probability distribution over the possible output tokens. Therefore, the sequence of tokens generated by a hidden Markov model gives some information about the sequence of states. A hidden Markov model can be considered a generalization of a mixture model where the hidden variables (or “latent” variables) which control the mixture component to be selected for each observation are related through a Markov process rather than independent of each other.

### Hidden or Latent States

In some embodiments, the hidden or latent states correspond to the presence or absence of at least one physiological condition. In some embodiments, the hidden or latent states correspond to the presence or absence of a disease, disorder, or condition in the subject. In some embodiments, the hidden or latent states correspond to a healthy condition. In some embodiments, the latent states correspond to clinical classifications of disease severity. In some embodiments, the clinical classifications of disease severity correspond to five latent states, representing cancer stages I, II, III, and IV, and a healthy (no cancer) state.

### Initial State Probabilities

In some embodiments, the hidden Markov model comprises initial state probabilities. Initial state probabilities may be set to any constant values determined to be appropriate based on the population from which a subject is sampled. In some embodiments, the prevalence of the disease, disorder, or condition or healthy condition in the population from which a subject is selected may be used to determine the prior probabilities of starting in each latent state. For example, if the prevalence of a rare disease is 1 in 10,000 individuals and the application of the hidden Markov model is to detect the presence of disease in asymptomatic individuals (i.e., individuals with average disease risk), the initial state probabilities may be set such that the probability of starting in the disease state is 1/10,000 and the probability of starting in the healthy state is 9,999/10,000.

In some embodiments, if the prevalence of the disease is unknown or if the human subject is at elevated risk, flat priors may be used as initial state probabilities. For example, the probability of starting in the disease state may be set to 0.5, and the probability of starting in the healthy state may similarly be set to 0.5.

### Transition Probabilities

In some embodiments, the hidden Markov model comprises or consists of a transition matrix comprising or consisting of transition probabilities. In some embodiments, the transition probabilities are set to specific and fixed constants. For example, constant values may be set to 0.9999, 0.999, 0.99, or 0.9 for transitioning from one state into the same state at the next observation; and 0.0001, 0.001, 0.01, or 0.1 for transitioning from one state into a different state at the next observation. In some embodiments, transition probabilities are set to arbitrary initial values (i.e., an initial guess) and then retrained and updated in an iterative process until some stopping criteria are met.

In some embodiments, the likelihood of the transition probability parameters is maximized with an algorithm. In some embodiments, the algorithm iterates until the difference in likelihood values between iterations is smaller than some small value epsilon.

In some embodiments, the algorithm comprises or consists of the Forward-Backward algorithm. The forward-backward algorithm is an inference algorithm for hidden Markov models which computes posterior marginals of all hidden state variables given a sequence of observations/emissions, i.e. it computes, for all hidden state variables, the distribution (See, Binder, J, Murphy, K., and Russell, S. Space-Efficient Inference in Dynamic Probabilistic Networks. Intik Joint Conf. on Artificial Intelligence, 1997, which is incorporated by reference in its entirety herein, including any drawings). The algorithm makes use of the principle of dynamic programming to efficiently compute the values that are required to obtain the posterior marginal distributions in two passes. The first pass goes forward in time while the second goes backward in time; hence the name forward-backward algorithm. The inference task is usually called smoothing.

### Emission Probabilities

In some embodiments, the hidden Markov model comprises or consists of emission probabilities. In some embodiments, the hidden Markov model emits endpoint counts at genomic coordinates. In some embodiments, maximum likelihood estimates for hidden states at a plurality of genomic positions from the hidden Markov model is obtained.

The emission probabilities are calculated with the use of the training distributions and a probability model. In some embodiments, a binomial probability model is used.

For example, for each coordinate c in a set of observations, the emission probability of observing k fragment endpoints at coordinate c, conditional on the latent state s and the training distribution iₛ is given by the equation:

$$Pr ( K = k \vert n , s , {t}_{s} ) = ( \begin{matrix}n \\ k\end{matrix} ) t \text{?} {( I - t \text{?} )}^{n - k}$$

$$\text{?} \text{indicates text missing or illegible when filed}$$

where n is the total number of endpoints in the vector in the testing dataset, and t_{s,c} is the frequency of endpoints at coordinate c the training distribution. Thus, the emission probability distribution for a given coordinate and state is the probability of observing a specific number of fragment endpoints out of a fixed number of trials (the sum total of all fragment endpoints in a region), conditional on the first training fragment endpoint map and the second training fragment endpoint map and the training distributions.

### Inference on the Disease, Disorder, or Condition

In some embodiments, maximum likelihood estimates are obtained with a Viterbi algorithm. A Viterbi algorithm may be employed by means of a computer program to create a vector of maximum likelihood estimate states for each analyzed region r. The Viterbi algorithm is a dynamic programming algorithm for finding the most likely sequence of hidden states—called the Viterbi path—that results in a sequence of observed events, especially in the context of Markov information sources and hidden Markov models (See, Viterbi A J (1967). Error bounds for convolutional codes and an asymptotically optimum decoding algorithm”. IEEE Transactions on Information Theory. 13 (2): 260-269, which is incorporated by reference herein in its entirety, including any drawings).

Each hidden or latent state is assigned an arbitrary numeric constant. For example, a healthy is assigned the constant 0 and cancer is assigned the constant 1.

For each analyzed region r having a length of L coordinates, the MLE states from the model are represented as a vector M:

Mᵣ=[m₁,m₂,m₃, . . . ,m_{L}]

In some embodiments, M̅ᵣ̅=Σᵢ₌₁^{L}mᵢ/L

In some embodiments, computing a summary statistic comprises or consists of creating a matrix, P, and computing the summary statistic with the matrix. In some embodiments, the summary statistic comprises or consists of a vector sum.

In some embodiments, matrix P is:

$$P = [ \begin{matrix}{\overline{M}}_{1 , 1} & ⋯ & {\overline{M}}_{1 , N} \\ ⋮ & ⋱ & ⋮ \\ {\overline{M}}_{R , 1} & ⋯ & {\overline{M}}_{R , N}\end{matrix} ]$$

Here, the R rows represent the regions in the analysis and the N columns represent the one or more individuals in the testing cohort.

### Inclusion of Labeled Samples

In some embodiments, one or more labeled samples—i.e., samples for which the true clinical status is known a priori—are also scored individually with the hidden Markov model. The same model parameters and training distributions that are selected for the test sample are used to analyze this set of labeled samples.

In some embodiments, the matrix P is:

$$P = [ \begin{matrix}{\overline{M}}_{1 , 1} & ⋯ & {\overline{M}}_{1 , N} & {\overline{M}}_{1 , N + 1} & ⋯ & {\overline{M}}_{1 , N + S} \\ ⋮ & ⋱ & & & & ⋮ \\ ⋮ & & ⋱ & & & ⋮ \\ ⋮ & & & ⋱ & & ⋮ \\ ⋮ & & & & ⋱ & ⋮ \\ {\overline{M}}_{R , 1} & ⋯ & {\overline{M}}_{R , N} & {\overline{M}}_{R , N + 1} & ⋯ & {\overline{M}}_{R , N + S}\end{matrix} ]$$

Here, the R rows represent the regions in the analysis and the N+S columns represent the N individuals in the testing cohort and the S individuals in the set of labeled samples included in the analysis.

### Inclusion of Full Matrix of Results

In some embodiments, matrix P is:

$$P = [ \begin{matrix}{m}_{1 , 1 , 1} & ⋯ & {m}_{1 , 1 , N} & {m}_{1 , 1 , N + 1} & ⋯ & {m}_{1 , 1 , N + S} \\ {m}_{2 , 1 , 1} & ⋱ & {m}_{2 , 1 , N} & ⋮ & ⋱ & ⋮ \\ ⋮ & & ⋮ & & & \\ {m}_{{L}_{1} , 1 , 1} & & & & & \\ {m}_{1 , 2 , 1} & & & & & \\ {m}_{2 , 2 , 1} & & & & & \\ ⋮ & & & & & \\ {m}_{{L}_{2} , 2 , 1} & & & & & \\ ⋮ & & & & & \\ {m}_{1 , R , 1} & & & & & \\ {m}_{2 , R , 1} & & & & & \\ ⋮ & & & & & \\ {m}_{{L}_{R} , R , 1} & ⋯ & {m}_{{L}_{R} , R , N} & {m}_{{L}_{R} , R , N + 1} & ⋯ & {m}_{{L}_{R} , R , N + S}\end{matrix} ]$$

Here, the MLE states from each genomic coordinate from each analyzed genomic region are included. Each element m_{x,y,z} represents the MLE state at coordinate x within a region y of length L_{y}, for sample z.

In some embodiments, MLE states are determined by the Viterbi algorithm. In some embodiments, the disease, disorder, or condition or physiological condition is diagnosed if the vector sum of MLE states exceeds a threshold value. In some embodiments, the disease, disorder, or condition or physiological condition is diagnosed if the vector median or mean is above a threshold value.

### Principal Components Analysis (PCA)

In some embodiments, the matrix P is decomposed into its principal components (PCs) by use of a computer program, according to the method of principal components analysis, to produce the decomposed matrix Q. Principal component analysis (PCA) is a statistical procedure that uses an orthogonal transformation to convert a set of observations of possibly correlated variables into a set of values of linearly uncorrelated variables called principal components.

In some embodiments, another matrix decomposition procedure is used to produce decomposed matrix Q. In some embodiments, singular value decomposition (SVD) is used.

In some embodiments, a subset of all PCs is retained and the remainder are discarded. In some embodiments, PCs are ranked according to the percentage of variance explained to produce a sorted list of PCs in which the first (top) element explains the highest percentage of the variance of the matrix P, and the last (bottom) element explains the lowest percentage of the variance of the matrix P. In some embodiments, top PCs are retained to produce a matrix. In some embodiments, the top 1, top 2, top 3, top 4, or top 5 PCs are retained to produce decomposed matrix Q.

### Support Vector Machine-Based Classification and Scoring

In some embodiments, some or all of decomposed matrix Q is used as input to train a support vector machine (SVM) to calculate maximum likelihood estimates. In some embodiments, the SVM is trained on a computer. In machine learning, support vector machines are supervised learning models with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall.

In some embodiments, labeled samples (i.e. samples for which the physiological condition is known) are included in matrix P and a decomposition matrix Q. Arbitrary class labels are assigned to each physiological condition—for example, 0 represents healthy state, and 1 represents disease state.

In some embodiments, determining if a summary statistic exceeds a threshold value comprises or consists of using an SVM to classify a test sample based on the location of the unlabeled sample in the multidimensional space defined by the SVM. The label assigned to the unlabeled sample is determined by which side of the decision boundary the unlabeled sample lies on. If the unlabeled sample falls on the “disease” side of the decision boundary, the “disease” label is applied; similarly, if the unlabeled sample falls on the “healthy” side of the decision boundary, the “healthy” label is applied.

In some embodiments, a score from the summary statistic is produced by calculating the Euclidean distance between a point representing the unlabeled sample and a threshold value. In some embodiments, distance is transformed to produce a score falling between two constants. For example, the constant 0 and 1 may be used. In some embodiments, scores close to 0 represent a higher probability that the sample is healthy and scores close to 1 represent a higher probability that the sample has the disease, disorder, or condition or physiological condition. In some embodiments, transformation occurs with a sigmoid function.

In some embodiments, a label is applied if the summary statistic exceeds a threshold value. A threshold value can be determined by one skilled in the art. In certain embodiments, a label is only applied if the percentage or absolute difference between a maximum calculated probability and a second-largest calculated probability exceeds a certain threshold. If the percentage or absolute difference falls below the threshold, no label is applied.

In some embodiments, many physiological conditions can be analysed simultaneously.

### XII. Computer Systems

Some embodiments comprise a computer system programmed to implement the methods provided herein. The computer system includes a central processing unit (“CPU”). The computer system also includes memory or memory location, electronic storage unit, communication interface for communicating with other systems, and peripheral devices, such as cache, other memory, data storage, and/or electronic display adapters. The memory, storage unit, interface, and peripheral devices are in communication with the CPU through a communication bus, such as a motherboard.

The storage unit can be a data storage unit. The computer system can be operatively coupled to a computer network. The network can be the Internet, an intranet and/or extranet, or an intranet and/or extranet that is in communication with the Internet. The network in some cases is a telecommunication and/or data network. The network can include one or more computer servers, which can enable distributed computing, such as cloud computing.

The CPU can execute a sequence of instructions, which can be embodied in a program or software. The instructions may be stored in the memory. The instructions can be directed to the CPU.

The computer system can include or be in communication with an electronic display that comprises a user interface for providing a report, which may include a diagnosis of a subject or a therapeutic intervention for the subject. The report may be provided to a subject, a health care professional, a lab-worker, or other individual.

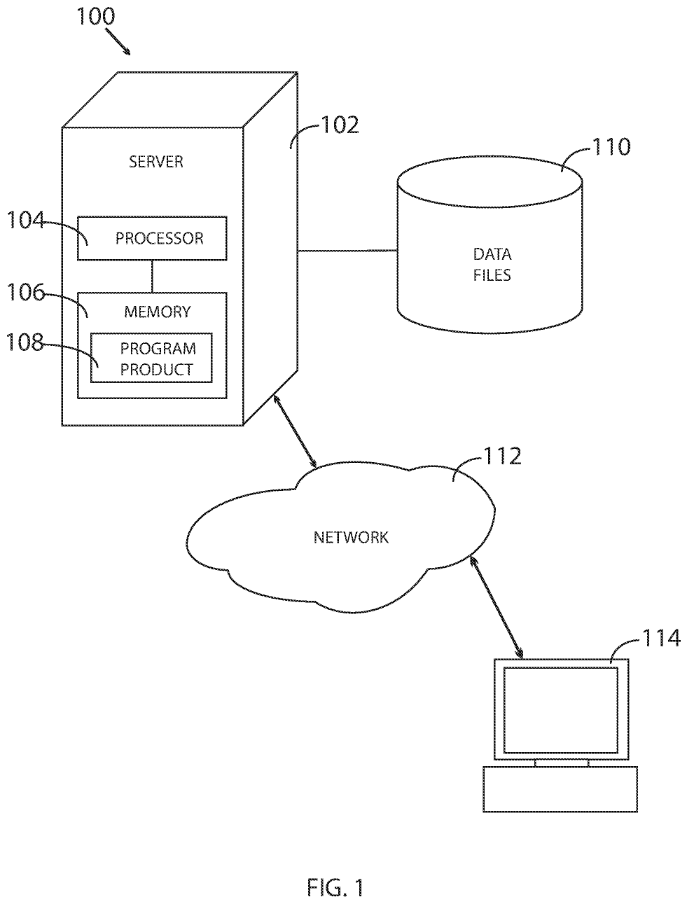

To illustrate, FIG. 1 provides a schematic diagram of an exemplary system suitable for use with implementing at least aspects of the methods disclosed in this application. As shown, system 100 includes at least one controller or computer, e.g., server 102 (e.g., a search engine server), which includes processor 104 and memory, storage device, or memory component 106, and one or more other communication devices 114 (e.g., client-side computer terminals, telephones, tablets, laptops, other mobile devices, etc.) positioned remote from and in communication with the remote server 102, through electronic communication network 112, such as the Internet or other internetwork. Communication device 114 typically includes an electronic display (e.g., an internet enabled computer or the like) in communication with, e.g., server 102 computer over network 112 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 100 also includes program product 108 stored on a computer or machine readable medium, such as, for example, one or more of various types of memory, such as memory 106 of server 102, that is readable by the server 102, to facilitate, for example, a guided search application or other executable by one or more other communication devices, such as 114 (schematically shown as a desktop or personal computer). In some aspects, system 100 optionally also includes at least one database server, such as, for example, server 110 associated with an online website having data stored thereon (e.g., control sample or comparator result data, indexed customized therapies, etc.) searchable either directly or through search engine server 102. System 100 optionally also includes one or more other servers positioned remotely from server 102, each of which are optionally associated with one or more database servers 110 located remotely or located local to each of the other servers. The other servers can beneficially provide service to geographically remote users and enhance geographically distributed operations.

As understood by those of ordinary skill in the art, memory 106 of the server 102 optionally includes volatile and/or nonvolatile memory including, for example, RAM, ROM, and magnetic or optical disks, among others. It is also understood by those of ordinary skill in the art that although illustrated as a single server, the illustrated configuration of server 102 is given only by way of example and that other types of servers or computers configured according to various other methodologies or architectures can also be used. Server 102 shown schematically in FIG. 1, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 100. As also understood by those of ordinary skill in the art, other user communication device 114 in these aspects, for example, can be a laptop, desktop, tablet, personal digital assistant (PDA), cell phone, server, or other types of computers. As known and understood by those of ordinary skill in the art, network 112 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 108 is optionally in the form of microcode, programs, cloud computing format, routines, and/or symbolic languages that provide one or more sets of ordered operations that control the functioning of the hardware and direct its operation. Program product 108, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 108 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 108 is optionally copied from the computer-readable medium to a hard disk or a similar intermediate storage medium. When program product 108, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one summary statistic, recommended treatment, and/or the like to be displayed (e.g., via communication device 114 or the like) and/or receive information from other system components and/or from a system user (e.g., via communication device 114 or the like).

In some aspects, program product 108 includes non-transitory computer-executable instructions which, when executed by electronic processor 104 perform at least: generating at least one first training fragment endpoint map from at least one first reference sample from one or more subjects with at least one first physiological condition, the at least one first training fragment endpoint map comprising measured frequencies of genomic locations of outer alignment coordinates, or a mathematical transformation thereof, within a reference genome for fragment endpoints from the at least one first reference sample; generating at least one second training fragment endpoint map from at least one second reference sample from one or more subjects with at least one second physiological condition, the at least one second training fragment endpoint map comprising measured frequencies of the genomic locations of outer alignment coordinates, or a mathematical transformation thereof, within the reference genome for fragment endpoints from the at least one second reference sample; and training a hidden Markov model with the at least one first training fragment endpoint map and the at least one second training fragment endpoint map.

System 100 also typically includes additional system components that are configured to perform various aspects of the methods described herein. In some of these aspects, one or more of these additional system components are positioned remote from and in communication with the remote server 102 through electronic communication network 112, whereas in other aspects, one or more of these additional system components are positioned local, and in communication with server 102 (i.e., in the absence of electronic communication network 112) or directly with, for example, desktop computer 114.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### XIII. Reports

Some embodiments comprise providing a report for the disease, disorder, or condition or physiological condition. An electronic report with scores can be generated to indicate diagnosis or prognosis. A diagnosis of a particular disease, disorder, or condition or physiological condition may then be made by a qualified healthcare practitioner. If an electronic report indicates there is a treatable disease, the electronic report can prescribe a therapeutic regimen or a treatment plan.

### XIV. Recommending and Providing Treatment

Some aspects and embodiments of the invention provide a method of recommending treatment for or providing treatment to a subject with a disease, disorder, or condition or a physiological condition. In some embodiments, the disease, disorder, or condition or physiological condition is cancer, normal pregnancy, a complication of pregnancy, myocardial infarction, inflammatory bowel disease, systemic autoimmune disease, localized autoimmune disease, allotransplantation with rejection, allotransplantation without rejection, stroke, and/or localized tissue damage. In some embodiments, the disease, disorder, or condition or first physiological condition is cancer. In some embodiments, the disease, disorder, or condition or physiological condition is lung adenocarcinoma, breast ductal carcinoma, or serous ovarian carcinoma. In some embodiments, the disease, disorder, or condition or physiological condition is colorectal cancer. In some embodiments, the method further comprises treating the identified disease, disorder, or condition or physiological condition in the subject.

### EXAMPLES

### Example 1

Frozen plasma specimens were obtained from 24 women with a confirmed diagnosis of high-grade serous ovarian cancer (HGSOC), 24 healthy women matched to the HGSOC patients on age and menopausal status, 8 women with benign ovarian tumors, and 8 women without ovarian cancer undergoing preparation for unrelated surgeries. A total of 1.0 mL of plasma was obtained from each patient. Cell-free DNA was purified from each specimen using the Qiagen Circulating Nucleic Acids kit according to the manufacturer's protocol. The yield of DNA was quantified by Qubit Fluorometer. Up to 10 ng of cfDNA from each specimen was used to create whole-genome, barcoded sequencing libraries. Each library was prepared with the Rubicon ThruPLEX Plasma-seq kit according to the manufacturer's protocol. Sequencing libraries were pooled and sequenced on an Illumina Novaseq instrument with the S4 flowcell. 2×100 cycle paired-end reads were obtained. Approximately 200 million fragments were sequenced from each specimen.

Reads were aligned to the human reference genome (version hg38) with the software bwa.

The two genomic coordinates representing the alignment endpoints of each properly paired fragment having mapping quality of at least 60 were determined using a custom software program. Only fragments having inferred lengths between 120 and 180 bp (inclusive) were considered.

The autosomal human reference genome was divided in silico into 3102 non-overlapping regions. Each region had a length of 1 megabase, with the exception of one region per chromosome whose length was defined by the number of coordinates remaining after dividing the length of the chromosome by 1,000,000. 11 healthy and 9 HGSOC datasets were randomly selected from the set of 48 samples to be used for training in a two-state hidden Markov model, where state 1 represents healthy and state 2 represents HGSOC. The hidden Markov model emissions probabilities were trained using the 20 training samples. The transition probabilities were:

Healthy→HGSOC=0.001

Healthy→Healthy=0.999

HGSOC→Healthy=0.001

HGSOC→HGSOC=0.999

The prior probabilities for states 1 and 2 were [0.5, 0.5], and these prior probabilities were identical for each of the 3,102 regions analyzed.

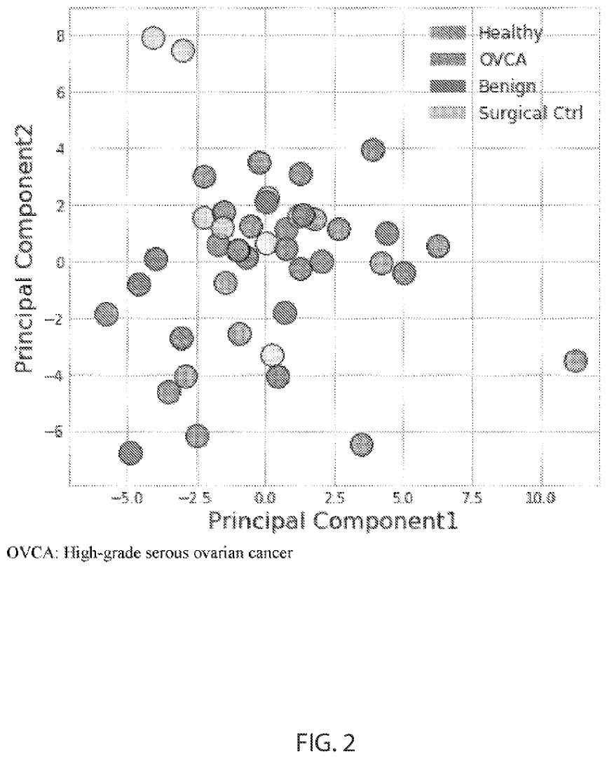

The trained model was applied to each of the 44 remaining samples, none of which had been used for training. The vectors of hidden Markov model output for each sample were combined into a matrix, with each column representing results for a single sample and rows representing the mean of the results for each region. The first two principal components of the resulting matrix are shown in FIG. 2.

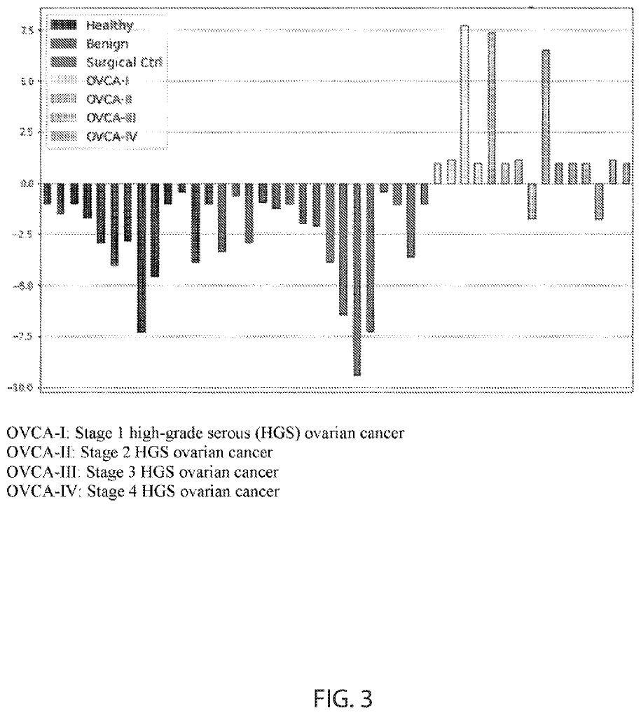

The first four principal components from the each of the labeled samples were then used to train a support vector machine (SVM). This trained SVM was then applied to the first four principal components of each of the blinded samples to generate a prediction (CRC or healthy) and an estimated probability or score for each sample. Scores close to 1.0 indicate a higher probability of HGSOC and scores close to 0.0 indicate a lower probability of HGSOC. The resulting scores are shown in FIG. 3.

The predictions on the blinded samples were evaluated by unblinding the samples after analysis had been performed and predictions had been generated. The resulting predictions correctly identified 13 of the 15 HGSOC samples as cancer and all 29 healthy samples as healthy, resulting in an overall accuracy of 95%.

### Example 2

Frozen plasma specimens were obtained from 27 individuals with a confirmed diagnosis of lung adenocarcinoma (LUCA), 32 women with a confirmed diagnosis of breast ductal carcinoma (BRCA), and 37 healthy individuals. A total of 3.0 mL of plasma was obtained from each patient. Cell-free DNA was purified from each specimen using the Qiagen Circulating Nucleic Acids kit according to the manufacturer's protocol. The yield of DNA was quantified by Qubit Fluorometer. Up to 15 ng of cfDNA from each specimen was used to create whole-genome, barcoded sequencing libraries. Each library was prepared with the Rubicon ThruPLEX Plasma-seq kit according to the manufacturer's protocol. Sequencing libraries were pooled and sequenced on an Illumina Novaseq instrument with the S4 flowcell. 2×100 cycle paired-end reads were obtained. Approximately 200 million fragments were sequenced from each specimen.

Reads were aligned to the human reference genome (version hg38) with the software bwa.

The two genomic coordinates representing the alignment endpoints of each properly paired fragment having mapping quality of at least 60 were determined using a custom software program. Only fragments having inferred lengths between 120 and 180 bp (inclusive) were considered.

Ten (10) non-overlapping genomic regions were used in the analysis. Only fragments having at least one outer alignment coordinate, also referred to as a fragment endpoint, falling within one of the genomic windows were retained. 10 Mb of sequence was targeted in silico in this manner. The regions are listed in Table 1.

TABLE 1

| | Start coordinate | End coordinate |
|---|---|---|
| Chromosome | (hg38) | (438) (hg38) |
| chr1 | 28000001 | 29000000 |
| chr2 | 162000001 | 163000000 |
| chr2 | 210000001 | 211000000 |
| chr3 | 185000001 | 186000000 |
| chr5 | 25000001 | 26000000 |
| chr10 | 77000001 | 78000000 |
| chr11 | 49000001 | 50000000 |
| chr11 | 110000001 | 111000000 |
| chr12 | 90000001 | 91000000 |
| chr14 | 34000001 | 35000000 |

18 healthy and 13 LUCA datasets were randomly selected from the set of samples to be used for training in a two-state hidden Markov model, where state 1 represents healthy and state 2 represents LUCA. The hidden Markov model emission probabilities were trained using the 31 training samples. The transition probabilities were:

Healthy→LUCA=0.001

Healthy→Healthy=0.999

The prior probabilities for states 1 and 2 were [0.5, 0.5], and these prior probabilities were identical for each of the regions analyzed.

The trained hidden Markov model was applied to each of the remaining 19 healthy and 14 LUCA samples, none of which had been used for training. A value of 1 was assigned to any genomic coordinate estimated to be in the LUCA state (state 2) and a value of 0 was assigned to any genomic coordinate estimated to be in the healthy state (state 1). The vectors of hidden Markov model output for each sample were combined into a matrix, with each column representing results for a single sample and rows representing the per-coordinate results for each of 20 targeted regions of the genome.

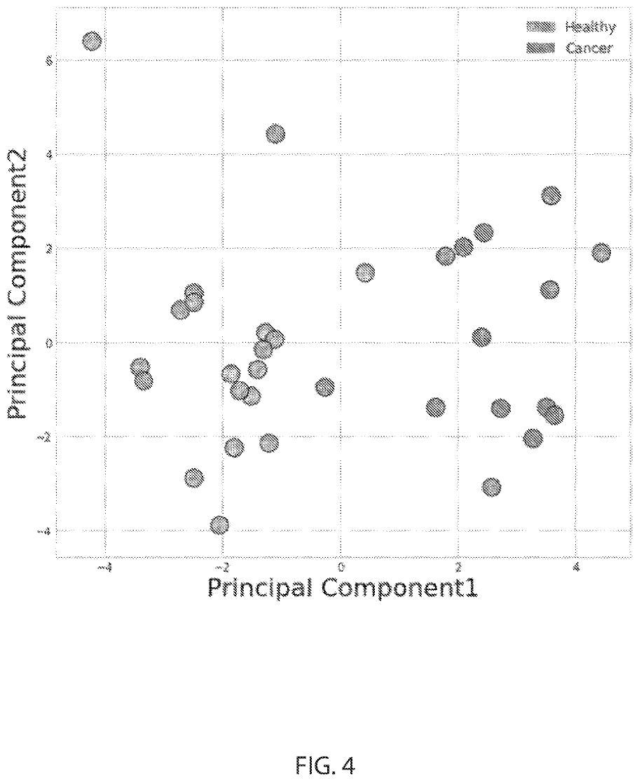

The first two principal components of this matrix are shown in FIG. 4.

Separately, 18 healthy and 16 BRCA datasets were randomly selected from the set of samples to be used for training in a two-state hidden Markov model, where state 1 represents healthy and state 2 represents BRCA. The hidden Markov model emissions probabilities were trained using the 34 training samples. The transition probabilities were:

Healthy→BRCA=0.001

Healthy→Healthy=0.999

BRCA Healthy=0.001

BRCA BRCA=0.999

The prior probabilities for states 1 and 2 were [0.5, 0.5], and these prior probabilities were identical for each of the twenty regions analyzed.

The trained model was applied to each of the remaining 19 healthy and 16 BRCA samples, none of which had been used for training. A value of 1 was assigned to any genomic coordinate estimated to be in the BRCA state (state 2) and a value of 0 was assigned to any genomic coordinate estimated to be in the healthy state (state 1). The vectors of hidden Markov model output for each sample were combined into a matrix, with each column representing results for a single sample and rows representing the per-coordinate results for each of 20 targeted regions of the genome.

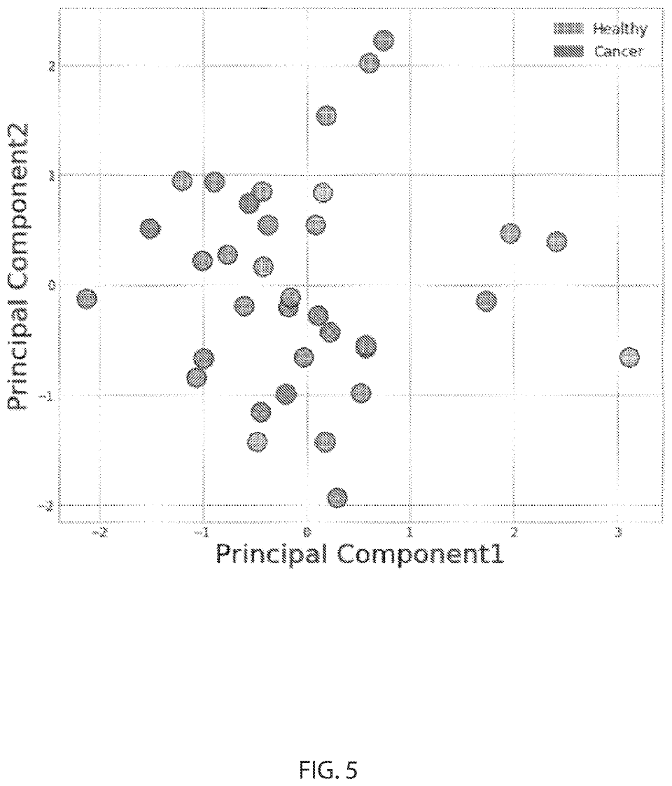

The matrix containing the results for the training samples was decomposed to its principal components. The first two principal components of this matrix are shown in FIG. 5. The first four principal components were selected and used to train a support vector machine (SVM). The remaining blinded samples were then projected into this reduced dimensional space and classified with the trained SVM.

### Example 3

Frozen plasma specimens were obtained from 27 individuals with a confirmed diagnosis of lung adenocarcinoma (LUCA), 33 women with a confirmed diagnosis of breast ductal carcinoma (BRCA), 10 individuals with a diagnosis of colorectal adenocarcinoma (CRCA), 6 individuals with a diagnosis of pancreatic ductal carcinoma (PACA), 2 men with a diagnosis of prostate cancer (PRCA), 8 individuals with a diagnosis of leukemia (LEUK), 8 individuals with a diagnosis of lymphoma (LYMP), 8 individuals with a diagnosis of myeloma (MYEL), and 48 healthy individuals. A total of 3.0 mL of plasma was obtained from each patient. Cell-free DNA was purified from each specimen using the Qiagen Circulating Nucleic Acids kit according to the manufacturer's protocol. The yield of DNA was quantified by Qubit Fluorometer. Up to 15 ng of cfDNA from each specimen was used to create whole-genome, barcoded sequencing libraries. Each library was prepared with the Rubicon ThruPLEX Plasma-seq kit according to the manufacturer's protocol. Sequencing libraries were pooled and sequenced on an Illumina Novaseq instrument with the S4 flowcell. 2×100 cycle paired-end reads were obtained. Approximately 200 million fragments were sequenced from each specimen.

Reads were aligned to the human reference genome (version hg38) with the software bwa. Reads were removed from the analysis if one or more of the following conditions were met: the read was a PCR or optical duplicate, the two reads of the read-pair were mapped to different chromosomes, or the orientation of the two reads of the read-pair were incorrect.

The two genomic coordinates representing the alignment endpoints of each properly paired fragment having mapping quality of at least 60 were determined using a custom software program. Only fragments having inferred lengths between 120 and 180 bp (inclusive) were considered.

From the full set of samples, one group comprising 14 healthy samples (“healthy”), and another group comprising 6 BRCA samples, 2 CRCA samples, 2 LEUK sample, 1 PRCA sample, 8 LUCA samples, 1 LYMP sample, 3 MYEL sample, and 1 PACA sample (“cancer mix”) were randomly selected to be used for training in a two-state hidden Markov model, where state 1 represents healthy and state 2 represents cancer. The hidden Markov model emission probabilities were trained using the two groups of training samples. The transition probabilities were:

Healthy→Cancer mix=0.001

Healthy→Healthy=0.999

Cancer mix→Healthy=0.001

Cancer mix→Cancer mix=0.999

The prior probabilities for states 1 and 2 were [0.5, 0.5].

The trained model was applied to each of the remaining samples that had not been used for training (“test samples”). A value of 1 was assigned to any genomic coordinate estimated to be in the cancer mix state (state 2) and a value of 0 was assigned to any genomic coordinate estimated to be in the healthy state (state 1).

From this set of test samples, 10 healthy, 3 CRCA, 1 LYMP, 1 MYEL, 1 LEUK, 4 BRCA, 3 LUCA, and 1 PACA were selected to be unblinded—i.e., the true label of each sample was known. The vectors of hidden Markov model output for each unblinded test sample were combined column-wise into a matrix, with each column representing results for a single sample and each row representing the result for a genomic coordinate. This matrix was then subjected to principal components analysis, and the top four principal components were retained.

These top four principal components from each unblinded sample were used to train a two-class linear discriminant analysis (LDA) model. In this LDA model, class 1 represented the healthy state, and class 2 represented the cancer mix state.

Each remaining test sample—i.e., each sample not used in either the hidden Markov model training or the LDA training—was treated as blinded. The vectors of the hidden Markov model output for each of the blinded test sample were combined column-wise into a matrix with each column representing results for a single sample and each row representing the result for a genomic coordinate. These results were then projected into the same principal component space defined by the unblinded samples; as before, the top four principal components were retained.

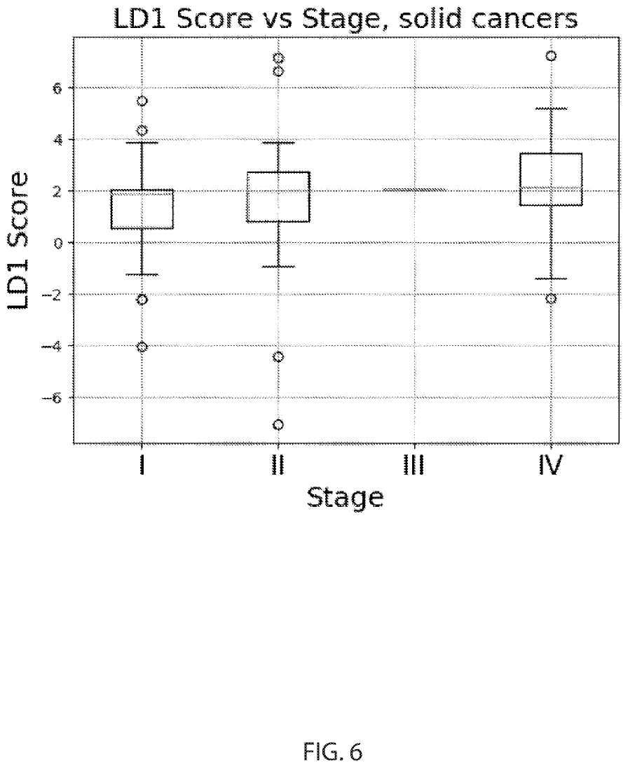

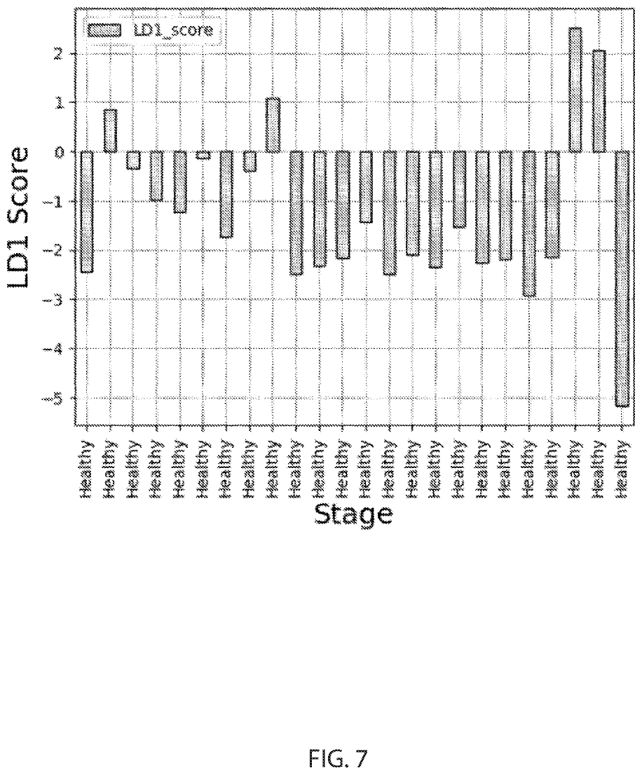

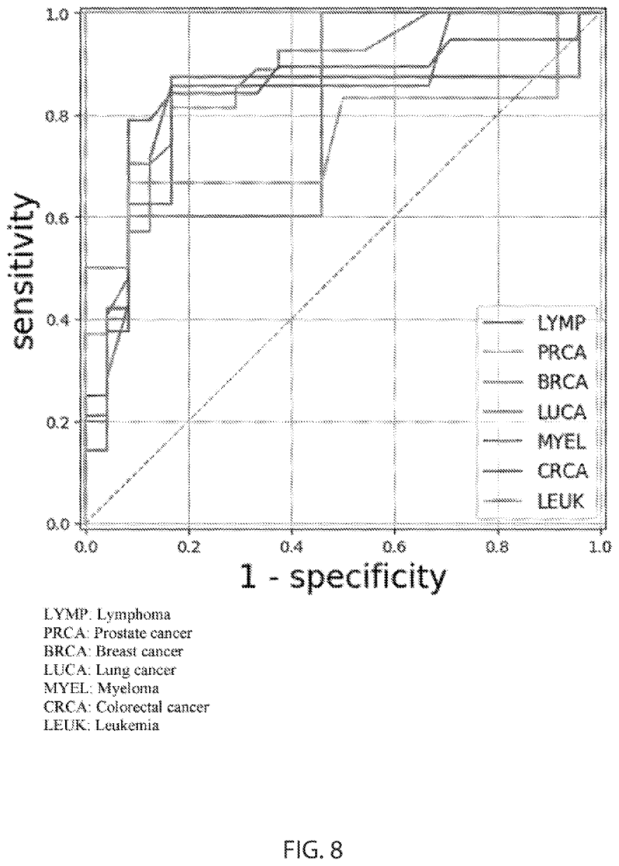

These top four principal components for each blinded test sample were finally used to make predictions about each sample's true disease class using the trained LDA model. For each blinded sample, a 1-dimensional linear discriminant score (“LD1 score”) was calculated. To determine the classification accuracy of the model, the unblinded samples were used to determine which side of the decision boundary represented the healthy samples. The LD1 scores for all solid tumor types, stratified by stage, are shown in FIG. 6. The LD1 scores for all healthy controls are shown in FIG. 7. The receiver operating characteristic curve for each tumor type, determined by sliding the classification decision boundary stepwise from the minimum to the maximum LD1 score and calculating the sensitivity and specificity of each resulting hypothetical classifier, is shown in FIG. 8.

### Example 4

Targeted sequencing data of cell-free DNA fragments purified from plasma samples from 150 individuals, including 83 cancer-free individuals and 67 individuals with a clinical diagnosis of colorectal adenocarcinoma, was obtained.

From this collection of datasets, 25 of the cancer-free samples and 20 of the colorectal cancer samples were randomly selected. The disease status of the samples in this set was unblinded. These labeled samples are referred to as “Training Set 2” in this example.

The disease status of the remaining 58 cancer-free samples and 47 cancer samples was blinded and is referred to as the “Test Set” in this example.

For each of the samples in Training Set 2 and the Test Set, a testing fragment endpoint map was created, as described herein, by tallying the genomic locations of the outer alignment coordinates within the human reference genome for each sample. In this example, only those coordinates within the human genome that were targeted by the assay were retained. Separately, healthy and cancer training fragment endpoint maps were constructed from targeted sequencing data of cell-free DNA fragments from plasma samples from 33 additional cancer-free individuals and 31 additional individuals with a clinical diagnosis of colorectal cancer, respectively. The same set of targeted coordinates mentioned above were represented in these training fragment endpoint maps.

Each of the samples in the Test Set and in Training Set 2 were individually analyzed with a hidden Markov model. Prior probabilities for each of the two disease states (healthy or cancer) were set to equal values of 0.5. A grid of possible transition probability values, ranging from 0.5 to 0.9999 for transitions from state s at coordinate t to state s at coordinate t+1, was evaluated, and the final probability values were selected by maximum likelihood.

The vectors of hidden Markov model output for each sample were combined into a matrix whose columns represent results for a single sample and whose rows represent the mean of the per-coordinate results for one of the targeted regions.

The first three principal components of each of the Training Set 2 samples were selected to train a logistic regression model. This trained model was then used to make predictions on each of the samples in the Test Set. Using a threshold of 0.5, samples in the Test Set were classified as either “colorectal cancer” (for values greater than 0.5) or “cancer-free” (for values less than 0.5).

In total, 51 of the 58 cancer-free samples in the Test Set were correctly identified, and 40 of the 47 colorectal cancer samples in the Test Set were correctly identified, resulting in specificity of 88% and sensitivity of 85%.

All publications and patent applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof.

1. A method of identifying a physiological condition in a subject, the method comprising:
a. providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
b. providing at least one first training fragment endpoint map from at least one first reference sample from one or more subjects with at least one first physiological condition, the at least one first training fragment endpoint map comprising measured frequencies of the genomic locations of outer alignment coordinates, or a mathematical transformation thereof, within the reference genome for fragment endpoints from the at least one first reference sample;
c. providing at least one second training fragment endpoint map from at least one second reference sample from one or more subjects with a second at least one second physiological condition, the at least one second training fragment endpoint map comprising measured frequencies of the genomic locations of outer alignment coordinates, or a mathematical transformation thereof, within the reference genome for fragment endpoints from the at least one second reference sample;
d. training a hidden Markov model with the at least one first training fragment endpoint map and the at least one second training fragment endpoint map;
e. obtaining maximum likelihood estimates for hidden states at a plurality of genomic positions from the hidden Markov model for the sample;
f. computing a summary statistic of the maximum likelihood estimates for the sample;
g. comparing the summary statistic to a threshold value; and
h. identifying the at least one first physiological condition in the subject if the summary statistic exceeds the threshold value.

2. The method of claim 1, wherein fragment endpoints from the sample, the at least one first reference sample, and/or the at least one second reference sample comprise or consist of cfDNA fragment endpoints.

3. The method of claim 2, wherein the at least one second physiological condition is a healthy human state.

4. The method of claim 3, wherein the at least one first physiological condition is cancer, normal pregnancy, a complication of pregnancy, myocardial infarction, inflammatory bowel disease, systemic autoimmune disease, localized autoimmune disease, allotransplantation with rejection, allotransplantation without rejection, stroke, and/or localized tissue damage.

5. The method of claim 4, wherein the at least one first physiological condition is cancer.

6. The method of claim 5, wherein the at least one first physiological condition is lung adenocarcinoma, breast ductal carcinoma, or serous ovarian carcinoma.

7. The method of claim 1, wherein the sample comprises or consists of whole blood, peripheral blood plasma, urine, or cerebral spinal fluid.

8. The method of claim 7, wherein the sample comprises or consists of plasma samples.

9. The method of claim 8, wherein the at least one first training fragment endpoint map and/or the at least one second training fragment endpoint map consist of positions or spacing of nucleosomes and/or chromatosomes, positions of transcription start sites and/or transcription end sites, positions of binding sites of at least one transcription factor, and/or positions of nuclease hypersensitive sites.

10. The method of claim 9, wherein the subject is human.

11. The method of claim 10, further comprising recommending treatment for or treating the at least one first physiological condition.

12. A method of identifying or diagnosing a disease, disorder, or condition in a subject, the method comprising:
a. providing a testing fragment endpoint map from a sample from the subject, the testing fragment endpoint map comprising measurements of the genomic locations of the outer alignment coordinates, or a mathematical transformation thereof, within a reference genome for at least some fragment endpoints;
b. providing at least one first training fragment endpoint map from at least one first reference sample from one or more subjects with a disease, disorder, or condition, the at least one first training fragment endpoint map comprising measured frequencies of the genomic locations of outer alignment coordinates, or a mathematical transformation thereof, within the reference genome for fragment endpoints from the at least one first reference sample;
c. providing at least one second training fragment endpoint map from at least one second reference sample from subjects not having the disease, disorder, or condition, the at least one second training fragment endpoint map comprising measured frequencies of the genomic locations of outer alignment coordinates, or a mathematical transformation thereof, within the reference genome for fragment endpoints from the at least one second reference sample;
d. training a hidden Markov model with the at least one first training fragment endpoint map and the at least one second training fragment endpoint map;
e. obtaining maximum likelihood estimates for hidden states at a plurality of genomic positions from the hidden Markov model for the sample;
f. computing a summary statistic of the maximum likelihood estimates for the sample;
g. comparing the summary statistic to a threshold value; and
h. identifying or diagnosing the disease, disorder, or condition in the subject if the summary statistic exceeds the threshold value.

13. The method of claim 12, wherein fragment endpoints from the sample, the at least one first reference sample, and/or the at least one second reference sample comprise or consist of cfDNA fragment endpoints.

14. The method of claim 13, wherein the disease, disorder, or condition is cancer, normal pregnancy, a complication of pregnancy, myocardial infarction, inflammatory bowel disease, systemic autoimmune disease, localized autoimmune disease, allotransplantation with rejection, allotransplantation without rejection, stroke, and/or localized tissue damage.

15. The method of claim 14, wherein the disease, disorder, or condition is cancer.

16. The method of claim 15, wherein the cancer is lung adenocarcinoma, breast ductal carcinoma, or serous ovarian carcinoma.

17. The method of claim 16, wherein the sample comprises or consists of whole blood, peripheral blood plasma, urine, or cerebral spinal fluid.

18. The method of claim 17, wherein the sample comprises or consists of plasma samples.

19. The method of claim 18, wherein the at least one first training fragment endpoint map and/or the at least one second training fragment endpoint map consist of positions or spacing of nucleosomes and/or chromatosomes, positions or transcription start sites and/or transcription end sites, positions of binding sites of at least one transcription factor, and/or positions of nuclease hypersensitive sites.

20. The method of claim 19, wherein the subject is human.

21-39. (canceled)
